(19) United States
(12) Patent Application Publication
(10) Pub. No.: US 2026/0110010 A1
(43) Pub. Date: Apr. 23, 2026

(54) Enzyme Based System for Production of Messenger RNA with Increased Transfection Efficiency

(71) Applicant: ImmunityBio, Inc., Culver City, CA (US)

(72) Inventors: Brett Morimoto, Culver City, CA (US); Kayvan Niazi, Culver City, CA (US); Annie Shin, Culver City, CA (US); Philip T. Liu, Culver City, CA (US); Lise Geissert, Culver City, CA (US)

(21) Appl. No.: 19/339,991

(22) Filed: Sep. 25, 2025

Related U.S. Application Data
(63) Continuation of application No. 17/931,352, filed on Sep. 12, 2022, now Pat. No. 12,448,638.
(60) Provisional application No. 63/244,990, filed on Sep. 16, 2021.

(51) Int. Cl. C12P 19/34; C12N 9/12

(52) U.S. Cl. CPC C12P 19/34; C12N 9/1241; C12N 9/1247; C12Y 207/07019

(57) ABSTRACT

A method for the production of proteins used in the in vitro transcription (IVT) of messenger RNA (mRNA), wherein the proteins are evaluated for purity and efficacy by the efficiency with which mRNA synthetically derived therefrom, subsequently transfects cells and produces encoded proteins.

### CROSS-REFERENCE TO RELATED APPLICATIONS

This application claims the benefit of priority under 35 U.S.C. § 119(e) to U.S. Provisional Patent Application No. 63/244,990, filed Sep. 16, 2021. The entire disclosure of U.S. Provisional Patent Application No. 63/244,990 is incorporated herein by reference.

### REFERENCE TO A SEQUENCE LISTING

This application contains a Sequence Listing submitted electronically as an ST.26 XML file format. The file, named “PAT005268_Sequence_Listing.xml”, has a size of 64000 bytes, and was created on 12 Sep. 2022. The information contained in the ST.26 XML file is incorporated herein by reference in its entirety pursuant to 37 CFR § 1.52(e)(5).

### FIELD

The present disclosure concerns production of synthetic messenger RNA (mRNA) via in vitro transcription (IVT) using a novel and cost effective method of enzyme production.

### BACKGROUND

Recent success in messenger RNA-based vaccines in combatting the COVID-19 pandemic has validated the efficacy of RNA therapeutics and highlighted the need for efficient methods of mRNA production. Optimization of the conditions of mRNA production would target better yields of the core proteins involved in mRNA synthesis, increased mRNA purity, and improved assay methods for assessing mRNA quantity and quality. Optimized proteins for mRNA synthesis would be helpful due to both increased efficiency of mRNA production as well as cost of the proteins themselves, which are commercially available. Key components of any strategy would involve optimized plasmid constructs for recombinant expression of the core proteins used in IVT, optimized protein production conditions, and optimized protein purification protocols. Hornblower & al. (2015) “Minding your caps and tails—considerations for functional mRNA synthesis” New England Biolabs White Paper describes an exemplary in vitro synthesis scheme for mRNA. There is still a need for optimized methods of production of clinical manufacturing grade enzymes for use in therapeutic mRNA synthesis.

### SUMMARY

Disclosed herein are apparatuses, systems and methods in the in vitro transcription of mRNA, wherein the process is optimized for the production of efficacious and inexpensive lots of clinically relevant agent for the treatment of human disease. The methods described herein relate to improvements in the production of the core proteins involved in in vitro transcription (IVT). The improvements include optimization in the expression vector sequences used for the bacterial production of proteins. The methods further include optimized conditions of production, the conditions including time and temperature. Finally, the methods include a means of evaluation of mRNA quality, where mRNA transfection and expression of encoded protein are assessed for quality of the protein components used in the IVT process.

In an embodiment, optimized bacterial expression vectors contain nucleotide sequences encoding the three IVT proteins are individually cloned into the expression vectors. In an aspect, a first DNA sequence, or insert, is cloned into a first bacterial expression vector, the first insert comprising the gene sequence for T7 RNA polymerase. The insert further comprises an arabinose promoter sequence upstream of the polymerase sequence. In an aspect, the insert is comprised of SEQ ID NO: 19. In an aspect, the codon optimized gene sequence for T7 RNA Polymerase is SEQ ID NO: 22.

In another aspect, a second gene sequence is cloned into a second bacterial expression vector, the insert comprising the gene sequences for the D1 and D12 subunits of the Vaccinia Virus Capping Enzyme (VVCE). The insert further comprises arabinose promoter sequences upstream on the D1 and D12 subunits. The insert further comprises ribosome binding sites (RBS) immediately downstream of the arabinose promoters. In an aspect, the insert is comprised of SEQ ID NO: 20. In an aspect, the codon optimized gene sequence for the D1 subunit of VVCE is SEQ ID NO: 23. In an aspect, the codon optimized gene sequence for the D12 subunit of VVCE is SEQ ID NO: 24.

In another aspect, a third insert is cloned into a third bacterial expression vector, the insert comprising the gene sequences for the Poly(A) polymerase. The insert further comprises an arabinose promoter sequence upstream of the Poly(A) polymerase sequence. In an aspect, the insert is comprised of SEQ ID NO: 21. In an aspect, the gene sequence for the Poly(A) polymerase is SEQ ID NO: 25.

In another aspect, bacteria contain expression vectors for protein expression and subsequent purification. In some cases, the conditions for protein growth and purification were enabled by modifications to the inserts which were cloned into the expression plasmids. Such modifications include placing an arabinose promoter upstream of the protein-encoding nucleotide sequence. Another modification involves placing two arabinose promoters upstream of VVCE, with one promoter upstream of the D1 subunit and the second promoter upstream of the D12 subunit. Further modifications include addition of nucleic acid sequences encoding His tags, wherein polymeric histidine is encoded in-frame with the protein sequence and placed at either the N or C terminus, and whereby proteins can be column purified after fermentation. Further modifications include addition of a Tobacco Etch Virus protease sequence, whereby the His tag sequence can be proteolytically removed subsequent to protein purification.

In an aspect, the purified proteins are sequentially added to an in vitro transcription reaction. First, the purified T7 RNA Polymerase is added to a reaction comprising linearized plasmid DNA encoding an mRNA transcript of interest. RNA is thereby transcribed from the linearized DNA via the action of the T7 RNA Polymerase. A second reaction comprises the RNA transcript from the first reaction, S-adenosyl Methionine, and purified VVCE. The RNA transcript is thereby capped at the N-terminal to generate cap 0 mRNA. A third reaction comprises capped mRNA from the VVCE reaction, purified poly(A) polymerase, and ATP, whereby a poly-adenylated tail is added to the 3′ end of the capped mRNA transcript.

Various objects, features, aspects and advantages will become more apparent from the following detailed description of preferred embodiments, along with the accompanying drawing figures in which like numerals represent like components.

### BRIEF DESCRIPTION OF THE DRAWINGS

FIG. 1 depicts structural features of mRNA. Figure from Vaccines (2020) 5:11. The critical quality attributes dictating mRNA expression performance are shown. Five critical quality attributes are identified that dictate efficient expression of a gene of interest. Three of these attributes require the use of enzymes: transcription (T7 RNA polymerase); capping (Guanylyltransferase); and Poly(A) tailing (Poly(A) polymerase). As of now, these three core enzymes need to be purchased for IVT.

FIG. 2: T7 polymerase expression construct. Leaky expression from the T7 promoting system caused toxic protein effects leading to mutations in gene sequence. Switching to a tightly regulated promoter allowed proposed cloning of the gene.

FIGS. 3A, 3B and 3C: Replacement of T7 promoter with arabinose promoter enables growth and protein expression in BL21 DE3 Clear Coli. Optimized growth conditions are shown. FIG. 3A shows the expression construct used. Leaky expression from the T7 promoting system caused toxic protein effects leading to mutations in gene sequence. Switching to a tightly regulated promoter allowed proposed cloning of the gene FIG. 3B shows protein gel results at 4 hour fermentation. FIG. 3C shows protein gel results at overnight (O/N) fermentation. In each Coomassie stained protein gel: lane 0 protein ladder; lane 1 cell pellet; lane 2 supernatant; lane 3 flow through; lane 4 wash; lane 5 wash 2; lane 6 imidazole wash; lane 7 elution 1; lane 8 elution 2; lane 9 elution 3; lane 10 elution 4, lane 11 elution 5; lane 12 elution 6; lane 13 elution 7.

FIGS. 4A and 4B: Demonstration of poor VVCE solubility when being expressed behind a T7 Promoter. FIG. 4A shows the expression construct used. Protein expression in BL21 DE3 Clear Coli. Results shown in the protein gel (FIG. 4B). In the Coomassie stained protein gel: lane 0 protein ladder; lane 1 cell pellet; lane 2 supernatant; lane 3 flow through; lane 4 wash; lane 5 imidazole wash; lane 6 elution 1; lane 7 elution 2; lane 8 elution 3; lane 9 elution 4, lane 10 elution 5. Solubility of the enzyme was hindered when being expressed behind a T7 Promoter.

FIGS. 5A and 5B: Improved solubility of VVCE D1 and D12 subunits when T7 promoters are replaced with arabinose promoters. FIG. 5A shows the expression construct used. Protein expression in BL21 DE3 Clear Coli (FIG. 5C). In the Coomassie stained protein gel: lane 0 protein ladder; lane 1 cell pellet; lane 2 supernatant; lane 3 flow through; lane 4 wash 1; lane 5 wash 2; lane 6 imidazole wash; lane 7 elution 1; lane 8 elution 2; lane 9 elution 3; lane 10 elution 4, lane 11 elution 5. Solubility of the enzyme was hindered when being expressed behind a T7 Promoter. Switching to the tightly regulated promoter and slowing down expression (18° C.) improved solubility.

FIGS. 6A and 6B: Addition of arabinose promoter enables growth and expression of insoluble Poly(A) Polymerase in BL21 DE3 Clear Coli. FIG. 6A shows the expression construct used. In the Coomassie stained protein gel (FIG. 6B): lane 0 protein ladder; lane 1 cell pellet; lane 2 supernatant; lane 3 flow through; lane 4 wash 1; lane 5 wash 2; lane 6 imidazole wash; lane 7 elution 1; lane 8 elution 2; lane 9 elution 3; lane 10 elution 4, lane 11 elution 5. Overexpressed protein is around 43 kD. Protein expression yielded a truncated enzyme with no solubility.

FIGS. 7A and 7B: Poly(A) Polymerase gene with UUG start codon enabled expression of protein with proper size and good solubility. Expression in BL21 DE3 Clear Coli. FIG. 7A shows the expression construct used. FIG. 7B western blot using anti-His. Lane 0 protein ladder; lane 1 cell pellet; lane 2 supernatant; lane 3 flow through; lane 4 wash 1; lane 5 wash 2; lane 6 imidazole wash; lane 7 elution 1; lane 8 elution 2; lane 9 elution 3. Protein expression yielded a truncated enzyme with no solubility. Addition of an unusual start codon and transcriptional regulation sequence allowed proper expression of enzyme.

FIG. 8: Transfection ready mRNA production workflow.

FIGS. 9A, 9B, 9C, 9D, 9E, and 9F: Flow Cytometry histogram analysis. Performance comparison of improved, Clear Coli-expressed versus commercial In Vitro Transcription enzymes. Detection of GFP expression in 293T Cells transfected with IVT GFP-mRNAs is shown in the plots. Plot shown is representative of triplicates.

FIGS. 10A and 10B: Performance comparison of improved, Clear Coli-expressed versus commercial In Vitro Transcription enzymes. Transfection result of IVT GFP-mRNAs produced using different enzymes. FIG. 10A shows the average GFP positive percentage. FIG. 10B shows the average geometric mean fluorescence intensity. In each graph, RNA sample is as follows: (1) untransfected 293t; (2) in vitro transcript without modifications; (3) in vitro transcript with 5′ cap; (4) uncapped HiScribe transcript with tail; (5) HiScribe transcript capped with ARCA; (6) HiScribe transcript capped with NEB VVCE; (7) mRNA transcript produced with in house enzymes (arrow).

### DETAILED DESCRIPTION

The disclosed components, compositions, systems, kits, and methods may be utilized for performing cell-free messenger RNA (mRNA) synthesis. Cell-free mRNA synthesis using in vitro transcription (IVT) exploits an ensemble of catalytic proteins prepared from the lysates of transfected bacterial cells. The purified proteins comprise an essential component of IVT reactions. A variety of methods exist for preparing proteins competent for cell-free mRNA synthesis, including those described herein.

All patents and published applications identified herein are incorporated by reference to the same extent as if each individual patent or application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### Definitions

In some embodiments, the numbers expressing quantities of ingredients, properties such as concentration, reaction conditions, and so forth, used to describe and claim certain embodiments are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to illuminate and does not pose a limitation on the scope of the claimed invention. No language in the specification should be construed as indicating any non-claimed element essential to the practice of the claimed invention.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### Expression Vectors

Expression vectors containing a nucleic acid encoding one or more mRNAs described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

### Messenger RNA

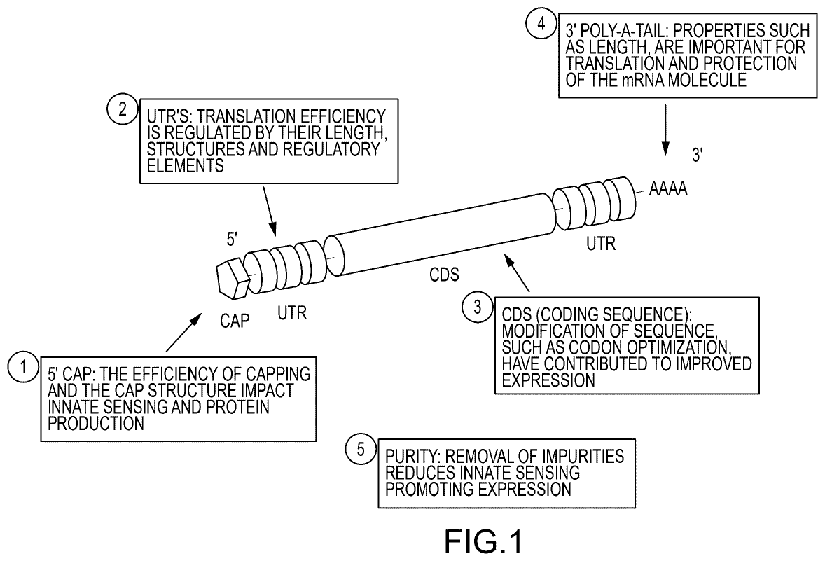

A surprising increase in mRNA quality, as determined by the expression levels of proteins expressed in mRNA transfected cells, results when commercially purchased enzymes T7 RNA polymerase, VVCE, and poly(A) polymerase were replaced with enzymes produced in-house in the mRNA production process. Enzyme production involves transfection of bacteria with plasmid expression vectors wherein the gene encoding a protein of interest is operably linked to a promoter sequence which drives expression of the transfected gene. FIG. 1 shows the key attributes of mRNA.

The promoter sequence may be any promoter suitable for driving protein expression in bacteria. In a preferred embodiment, the promoter is an arabinose promoter. Further engineering of genetic sequences inserted into the plasmid expression vector include the use of a tag by which a protein may be identified and/or purified. In a preferred embodiment, the gene encoding the protein of interest is linked to a sequence encoding a polymeric histidine (His) tag in-frame at either the 5′ or the 3′ end of the protein. The His tag may be separated from the gene of interest by a tobacco etch virus (TEV) protease recognition sequence, whereby TEV can be added to the protein to cleave off the His tag.

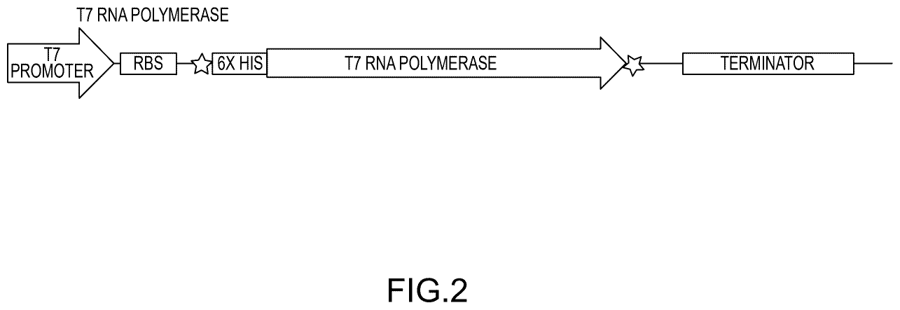

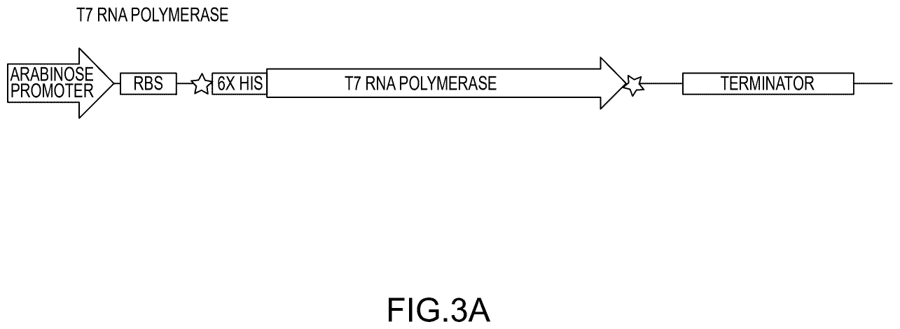

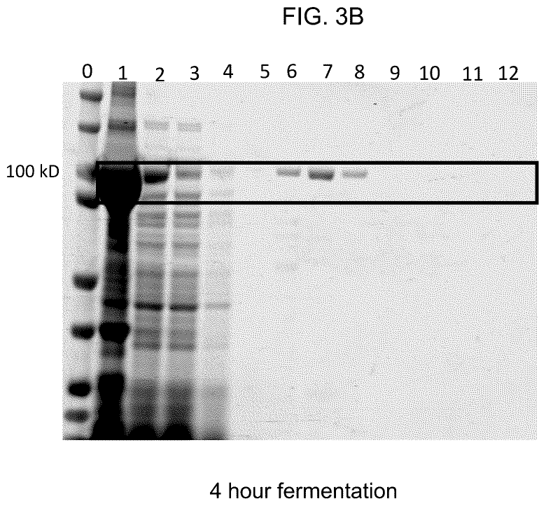

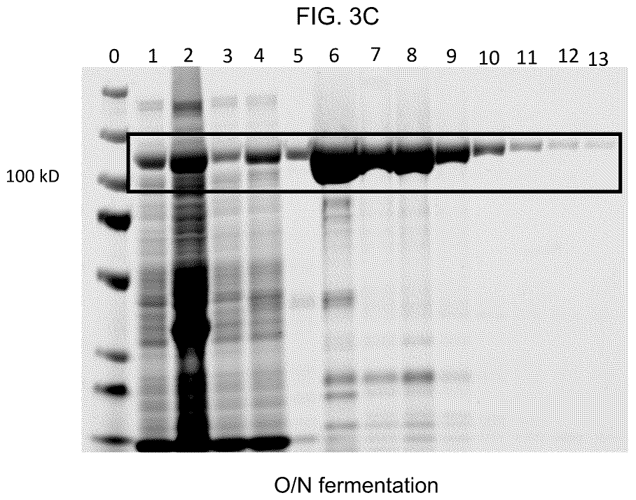

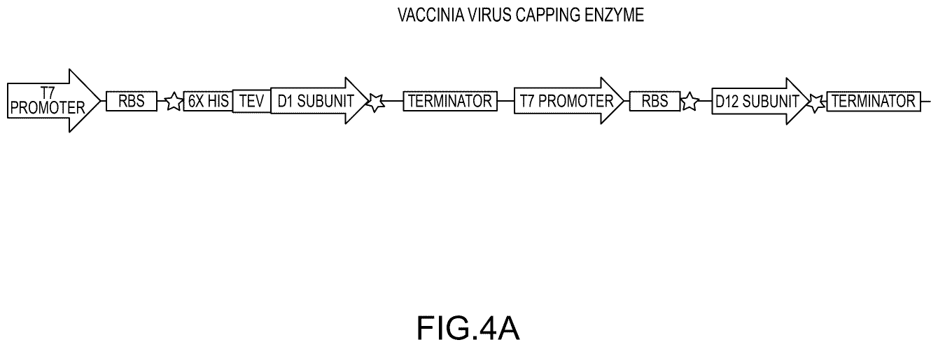

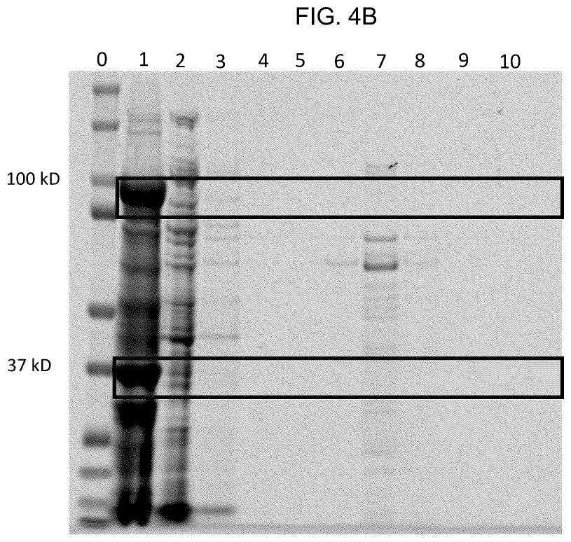

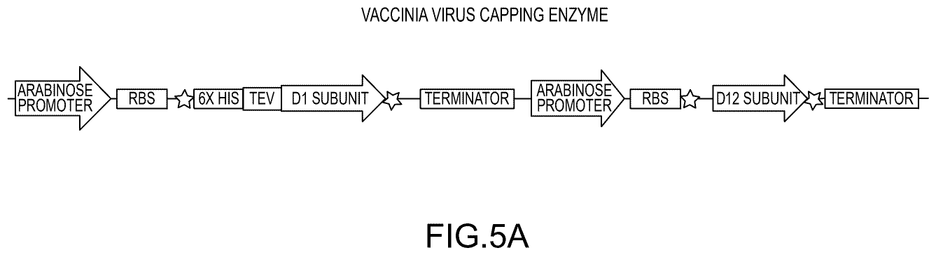

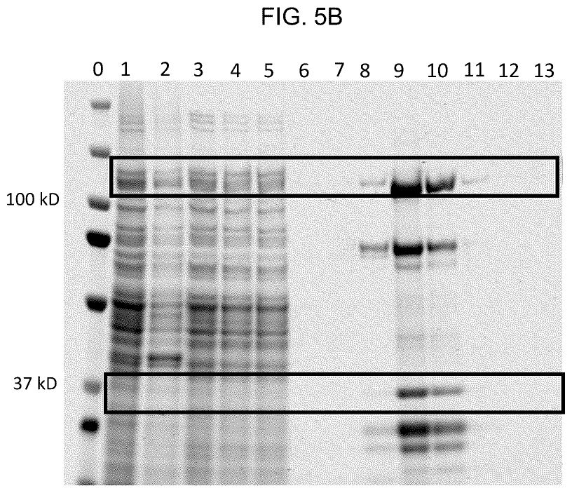

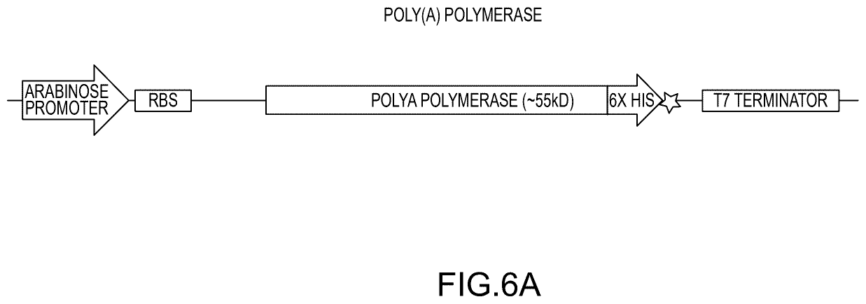

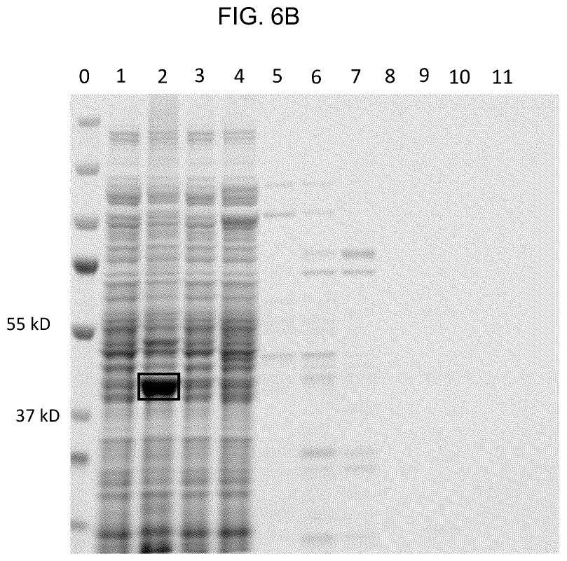

Replacement of the T7 promoter with an arabinose promoter driving expression of T7 RNA polymerase (FIG. 2 and FIGS. 3A-3C) was shown to enable growth of BL21 DE3 Clear Coli cells, and to drive heterologous protein expression therein. Increasing time of fermentation from 4 hours to overnight (18 hours) also increased protein expression. Similar replacement of T7 promoters driving expression the D1 and D12 subunits of VVCE with arabinose promoters enabled expression of the subunits (FIGS. 4A-4B and FIGS. 5A-5B). Performing the protein expression at 18° C. also improved protein expression.

A sequence for the production of an IVT protein may further be modified by the addition of an alternative start codon to the protein encoding sequence. In a preferred embodiment, an alternative start codon is linked to the N-terminus of poly(A) polymerase. This unusual UUG start codon of poly(A) polymerase was first postulated in Cao & Sarkar (Proc. Natl. Acad. Sci. USA. Vol. 89, pp. 10380-10384, November 1992). An N-terminal sequence (SEQ ID NO: 21) comprising the UUG start codon is encoded in-frame immediately upstream of the lysine at amino acid position 11 with respect to the wild type protein sequence of SEQ ID NO:25.

An expression plasmid insert sequence for the production of an IVT protein may further be modified by the use of novel promoter sequences. Surprisingly, an arabinose promoter significantly enhances the solubility of VVCE, and hence the yield of VVCE expressed in E. coli. Also surprising, the cloning itself of the T7 RNA polymerase and Poly(A) polymerase genes into expression vectors was enabled by placing an arabinose promoter upstream of the start codon of both proteins, as the use of a T7 promoter upstream of T7 RNA polymerase and poly(A) polymerase resulted in an inability to generate clonal expression plasmids which could successfully propagate in bacteria by standard techniques.

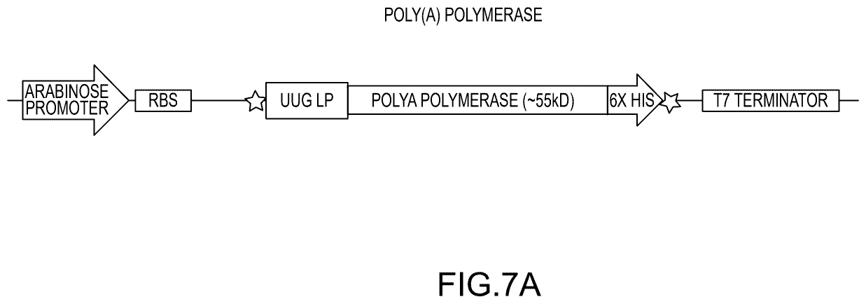

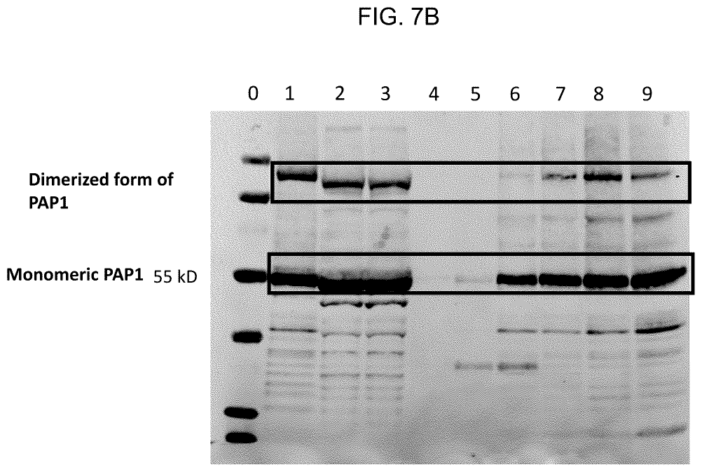

Disclosed herein are methods by which production of T7 RNA polymerase is optimized with respect to protein yield. After transfecting the expression plasmids into BL21 DE3 Clear Coli, the temperature of fermentation was reduced to 18° C. and the time was extended to 18 hours. FIG. 7 shows an increase in the yield of soluble T7 RNA polymerase when these fermentation conditions were applied.

Expression vectors containing a nucleic acid encoding one or more mRNAs described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

Having optimized the production of T7 RNA polymerase, VVCE, and poly(A) polymerase in bacteria, and having purified and characterized the proteins by standard methods, the proteins were applied in a sequential series of reactions whereby a linearized plasmid DNA containing a gene of interest is transcribed, capped, and a poly-adenosine tail is added. The plasmid into which the DNA has been inserted may be appropriate for propagation in viruses, prokaryotes, or eukaryotes.

Template DNA typically is propagated and stored as supercoiled plasmid DNA. Plasmid DNA is linearized through the action of one or more restriction enzymes, whereby the gene of interest is removed, or excised from the circular plasmid DNA. Typically unique restriction sites occur at the 5′ and 3′ ends of the gene of interest. The restriction sites may be the same or different. Any restriction enzymes can be used provided they do not recognize restriction sites within the gene of interest. The linearized DNA is then isolated via column purification, although other standard methods such as ethanol or isopropanol precipitation may be used. The purified linearized DNA comprising the gene sequence is then isolated by size determination and quantitated spectrophotometrically. One μG template DNA is then added to a reaction comprising nucleotide triphosphates (ATP, CTP, GTP, and UTP) and the purified T7 RNA polymerase. The reaction proceeds overnight at 37° C. in a thermal cycler. The RNA transcript is then column purified and quantitated via spectrophotometry.

Conversion of an RNA transcript to cap 0 RNA requires three sequential enzymatic steps: the removal of the 5′ terminal g-phosphate by RNA triphosphatase activity (TPase), the transfer of a GMP group to the resultant diphosphate 5′ terminus by RNA guanylyltransferase activity (GTase) and the modification of the N7 amine of the guanosine cap by guanine-N7 methyltransferase activity (MTase). Vaccinia Virus capping enzyme consists of D1 and D12 subunits, with all three enzymatic steps performed by the D1 subunit. The purified and quantitated RNA transcript (10 μg) is then added into a reaction comprising GTP, s-adenosyl methionine, and purified VVCE. The reaction then proceeds at 37° C. for one hour. The capped RNA is then column purified and spectrophotometrically quantitated.

Polyadenylation is the addition of a poly(A) tail to an mRNA transcript. The poly(A) tail is important for the nuclear export, translation and stability of mRNA. The final step in the in vitro production of mRNA is the addition of a poly(A) tail to the capped transcript. The capped mRNA (10 μg) transcript is added to a reaction comprising ATP and purified poly(A) polymerase, and the reaction proceeds for one hour at 37° C. The capped, polyadenylated mRNA transcript is then column purified and spectrophotometrically quantitated. The purified mRNA should be used immediately or frozen at −80 C or below.

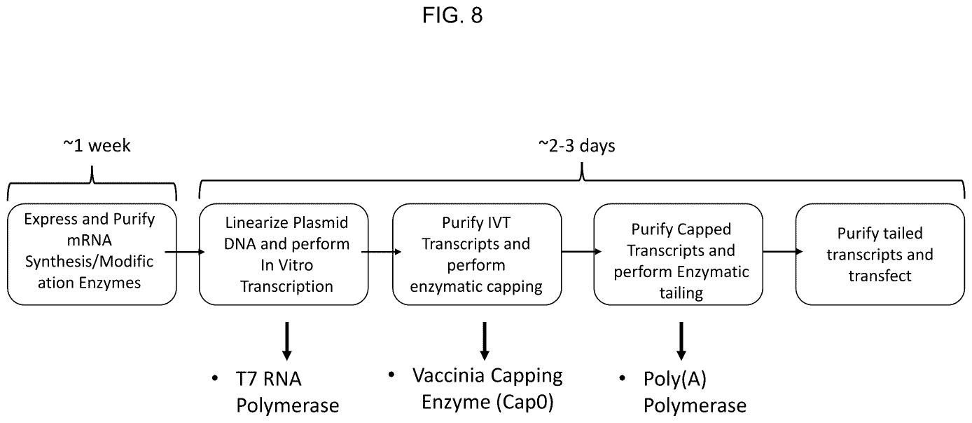

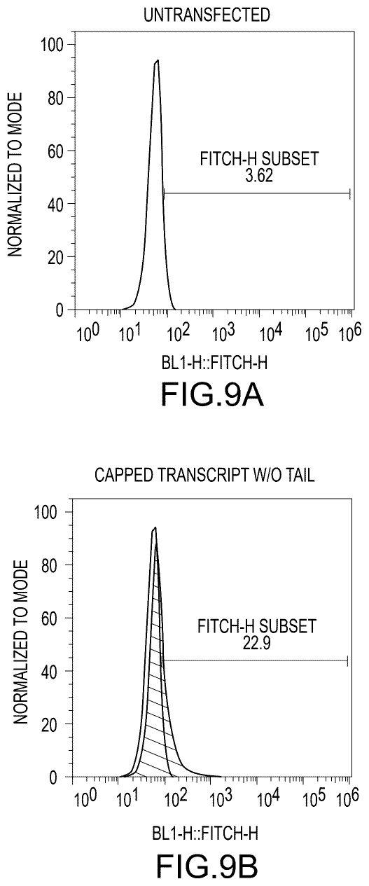

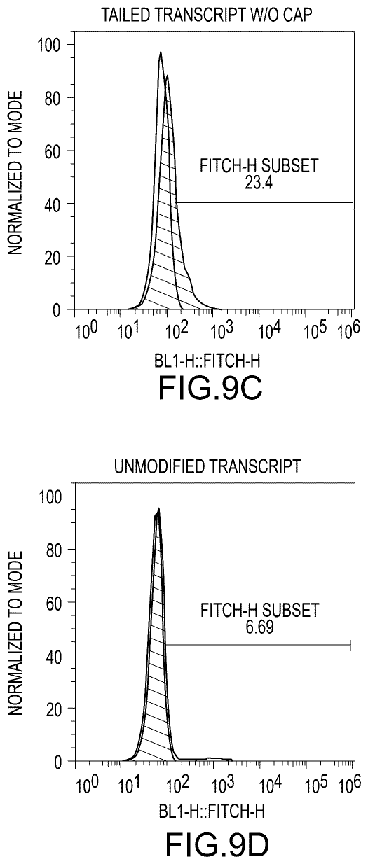

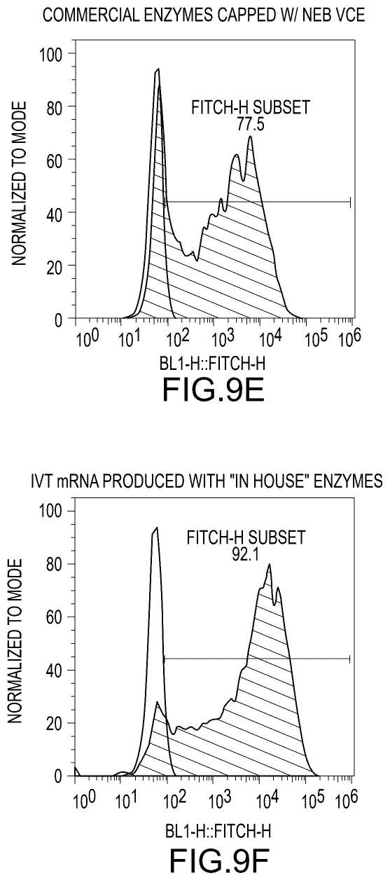

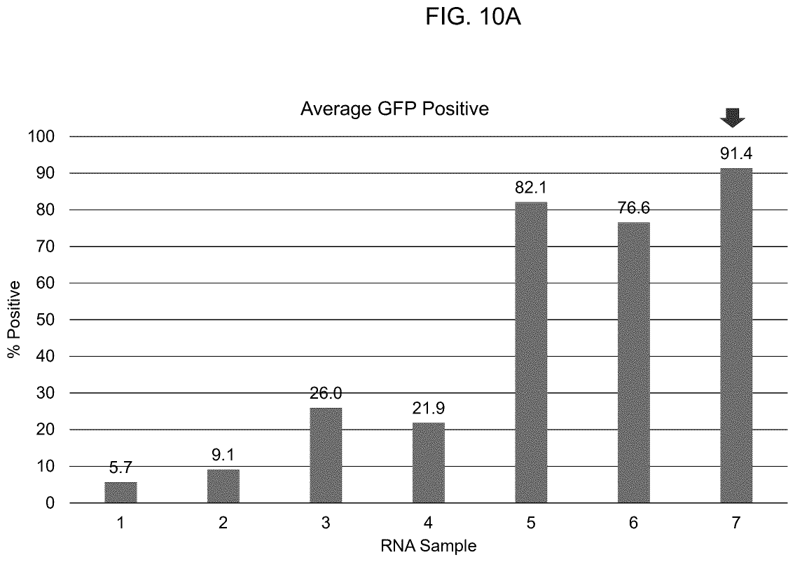

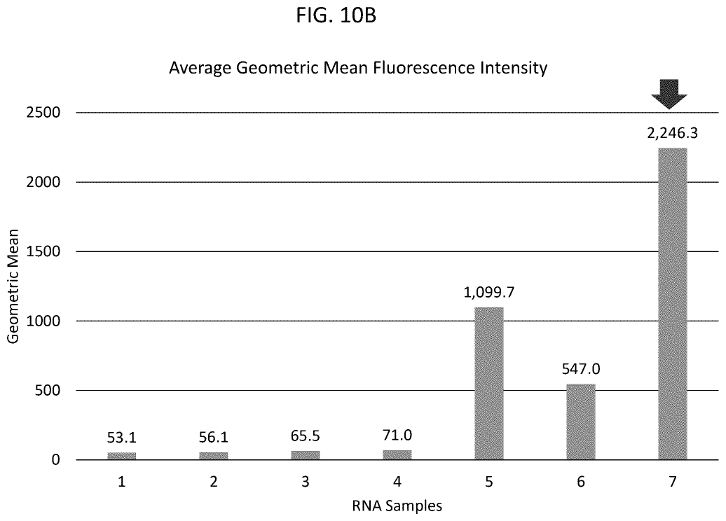

A summary of the mRNA workflow is shown in FIG. 8. mRNA derived thereby may be formulated for delivery to cells (in vitro transfection) or tissues (in vivo transfection). Any suitable formulation may be used. An exemplary nanoparticle formulation is described in U.S. Ser. No. 16/622,908, the contents of which are hereby incorporated by reference. Evaluation of delivery and expression of mRNA encoded proteins using standard methods, as presented in FIGS. 9A-9F wherein HEK293 cells are transfected and assessed via flow cytometry. FIGS. 10A and 10B show improved protein yield with mRNA produced by the methods described herein over commercially available RNA transcripts capped with commercially available VVCE protein from NEW ENGLAND BIOLABS®.

One should appreciate that the disclosed techniques provide many advantageous technical effects including increased production of IVT core proteins T7 RNA polymerase, Vaccinia Virus Capping Enzyme, and poly(A) polymerase, as well as increased yield of protein products derived from mRNA transcripts produced thereby.

### EXAMPLES

Preparation of Arabinose Promoter Insert. pBAD-DEST49 DNA plasmid was used as template for the PCR used to amplify the arabinose promoter. SEQ ID NO:1 (forward) and SEQ ID NO:2 (reverse) primers were used to amplify the arabinose promoter. The amplification mix comprised (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of pBAD-DEST49; 1 μL of PRIMESTAR® GXL Polymerase; and MILLI-Q® water to a final volume of 50 μL. The following conditions were used for the amplification of the Arabinose insert: 98° C. for 2 minutes; 98° C. for 20 seconds (×30 cycles); 55° C. for 15 seconds (×30 cycles); 68° C. for 90 seconds (×30 cycles); and 68° C. for 1 minute. The PCR product was purified using QIAQUICK® PCR spin columns.

Purified PCR products were then used in another PCR using primers SEQ ID NOs: 3 and 4. The amplification mix comprised (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of pBAD-DEST49; 1 μL of PRIMESTAR® GXL Polymerase; MILLI-Q® water to a final volume of 50 μL. The following conditions were used for the amplification of the Arabinose insert: 98° C. for 2 minutes; 98° C. for 20 seconds (×30 cycles); 60° C. for 15 seconds (×30 cycles); 68° C. for 90 seconds (×30 cycles); and 68° C. for 1 minute. Arabinose promoter insert with Gibson assembly handles was then gel extracted and purified using the QIAGEN® Gel Extraction kit.

BglIII Restriction Digest Protocol. pET22b Lactose induced expression system was digested using BglIII restriction enzyme purchased from NEW ENGLAND BIOLABS®. Two separate reactions were completed for a pET22b and BglIII restriction digest as follows (per reaction): 10 μL of Buffer 3.1; 3 μL BglIII restriction enzyme; 46 μL pET22b DNA; 41 μL of MILLI-Q® Water. Each reaction was then incubated in a thermal cycler at 37° C. for 2 hours. Following incubation, 500 μL of Buffer PB was added to each reaction. Buffer PB and Restriction digest mixture was added to two separate mini prep columns purchased from QIAGEN®. Columns were spun for 1 minute at 15000×g and flow-through was discarded. 1 mL of Buffer PE containing Ethanol was added to each column. Columns were again spun for 1 minute at 15000×g and flow-through was discarded. Columns were spun dry for 2 minutes at 15000×g. Columns then transferred to new microcentrifuge tubes. 50 μL of MILLI-Q® Water were added to each column and spun for 2 minutes at 15000×g.

Xba1 Restriction Digest Protocol. 50 μL of the purified and digested material was used in the digest using Xba1 with the following protocol (per reaction): 9 μL of CUTSMART® Buffer; 3 μL of Xba1; 50 μL of BglIII digested Material; and 28 μL of MILLI-Q® Water. Reactions were incubated in a thermal cycler at 37° C. for 2 hours. Antarctic Phosphatase was then added (10 μL/reaction of Antarctic Phosphatase Buffer & 2 μL/reaction of Antarctic Phosphatase). The reaction mixtures were then incubated at 37° C. for 1 hour. The digested vector was gel extracted and purified using the QIAGEN® gel extraction purification kit.

GIBSON ASSEMBLY® Protocol. 100 ng of the digested and purified vector was prepared for the reaction. 70 ng of the amplified and purified promoter was also prepared for the reaction. Each reaction contained only 0.1 pmols of total DNA used in the GIBSON ASSEMBLY®. Vector DNA and Insert DNA were mixed with MILLI-Q® water to a final volume of 10 μL. 10 μL of 2×NEB HIFI Assembly Master mix (obtained from NEW ENGLAND BIOLABS®) was then added to the reaction mixture. The prepared GIBSON ASSEMBLY® reactions were then incubated in a thermal cycler at 50° C. for 15 minutes. Following the incubation period, 8 μL of the reaction mixture were then transformed using NEB5α ultracompetent cells. Transformed cells with the arabinose promoter cloned into the pET22b expression plasmid were used to amplify the plasmid DNA which was then harvested using the QIAGEN® Maxi Prep Purification system.

Cloning of pBM100 for creation of T7 RNA Polymerase expression system. The RNA polymerase with the Gibson handle as well as the incorporated 6×His tag was PCR amplified using SEQ ID NOs: 5 and 6 as primers. The PCR reaction mixture comprised (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of RNA Polymerase DNA; and 1 μL of PRIMESTAR® GXL Polymerase. The PCR to amplify the RNA Polymerase insert was completed as follows: 98° C. for 2 minutes; 98° C. for 20 seconds (×30 cycles); 60° C. for 15 seconds (×30 cycles); 68° C. for 120 seconds (×30 cycles); and 68° C. for 1 minute. The completed PCR was then purified using QIAQUICK® PCR spin columns. 20 μg of pBM98 was used in a double restriction digest using the enzymes: Nde1 and Nco1-HF using the following reaction recipe (per reaction): 90 μL of CUTSMART® buffer; 1 μL of Nco1-HF; 1 μL of Nde1; 20 μg of RNA Polymerase DNA; and water to a final volume of 90 μL. The restriction digest was then incubated in a thermal cycler for 2 hours at 37° C. Antarctic Phosphatase was then added (10 μL/reaction of Antarctic Phosphatase Buffer and 2 μL/reaction of Antarctic Phosphatase). The reaction mixture was then incubated at 37° C. for 1 hour. The digested vector was then gel extracted and purified using the QIAGEN® gel extraction purification kit.

100 ng of the digested and purified vector was prepared for a GIBSON ASSEMBLY® reaction. 126 ng of the amplified and purified RNA Polymerase insert was also prepared for the reaction. No more than 0.1 pmols of total DNA was used in each GIBSON ASSEMBLY® reaction. Vector DNA and Insert DNA were mixed with MILLI-Q® water to a final volume of 10μ. 10 μL of 2×NEB HIFI Assembly Master mix (obtained from NEW ENGLAND BIOLABS®) was then added to each reaction mixture. The prepared GIBSON ASSEMBLY® reactions were incubated in a thermal cycler at 50° C. for 15 minutes. Following the incubation period, 8 μL of the reaction mixture were then transformed using NEB5α ultracompetent cells. Transformed cells with the T7 RNA Polymerase cloned into the Arabinose Promoted expression plasmid were used to amplify the plasmid DNA which was then harvested using the QIAGEN® Mini Prep Purification system.

Creation of Dual Arabinose Promoting Expression Plasmid. The dual expressing arabinose promoting system was created for cloning of the Vaccinia Virus Capping Enzyme D1, and D12 subunits. pET-Duet purified plasmid was digested with Xba1 using the following protocol (per reaction): 9 μL of CUTSMART® Buffer; 3 μL of Xba1; 10 μg pET-Duet plasmid; MILLI-Q® water to a final volume of 90 μL. Reactions were incubated in a thermal cycler at 37° C. for 2 hours. Antarctic Phosphatase was then added (10 μL/reaction of Antarctic Phosphatase Buffer & 2 μL/reaction of Antarctic Phosphatase). Reaction mixture was then incubated at 37° C. for 1 hour.

The digested vector was then gel extracted and purified using the QIAGEN® gel extraction purification kit. The arabinose promoter insert was then amplified out of pBM98 using SEQ ID NOs: 1 and 2 as the primers, with the following reaction mixtures (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of pBM98; 1 μL of PRIMESTAR® GXL Polymerase; and MILLI-Q® water to a final volume of 50 μL. The following conditions were used for the Amplification of the Arabinose insert: 98° C. for 2 minutes; 98° C. for 20 seconds (×30 cycles); 55° C. for 15 seconds (×30 cycles); 68° C. for 90 seconds (×30 cycles); and 68° C. for 1 minute. The PCR product was purified using QIAQUICK® PCR spin columns.

Purified PCR products were then used in another PCR using SEQ ID NOs: 7 and 8 as primers. This PCR product was used for the GIBSON ASSEMBLY® in order to create the Multiple cloning site #1 with the arabinose promoting system. 100 ng of the digested and purified vector was prepared for a GIBSON ASSEMBLY® reaction. 70 ng of the amplified and purified promoter was also prepared for the reaction. No more than 0.1 pmols of total DNA was used in the GIBSON ASSEMBLY®. Vector DNA and Insert DNA were mixed with MILLI-Q® water to a final volume of 10 μL. 10 μL of 2×NEB HIFI Assembly Master mix (obtained from NEW ENGLAND BIOLABS®) was then added to the reaction mixture. The prepared GIBSON ASSEMBLY® reactions were then incubated in a thermal cycler at 50° C. for 15 minutes. Following the incubation period, 8 μL of the reaction mixture were then transformed using NEB5α ultracompetent cells. Transformed cells with the arabinose promoter cloned into the pET-Duet dual expression plasmid were used to amplify the plasmid DNA which was then harvested using the QIAGEN® Midi Prep Purification system. The resulting plasmid was labeled as pBM122 to denote the presence of an arabinose promoter in MCS1 and a T7 promoter in the MCS2.

The newly constructed plasmid was then used to replace the downstream T7 promoter with another arabinose promoting insert. A dual restriction digest was complete on the prepared plasmid using the enzymes Bsrg1-HF and Nde1 with the following reaction mix (per reaction): 9 μL of CUTSMART® Buffer; 1.5 μL of Bsrg1-HIF; 1.5 μL of Nde1; 30 μg of pBM122; and MILLI-Q® water to a final volume of 90 μL.

Restriction digests were incubated at 37° C. for 1 hour. Antarctic Phosphatase was then added (10 μL/reaction of Antarctic Phosphatase Buffer & 2 μL/reaction of Antarctic Phosphatase). Reaction mixture was then incubated at 37° C. for 1 hour. The digested vector was then gel extracted and purified using the QIAGEN® gel extraction purification kit. The arabinose promoter was prepared with SEQ ID NOs: 1 and 2 as primers. PCR was completed using the following protocol (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of pBM98; 1 μL of PRIMESTAR® GXL Polymerase; and MILLI-Q® water to a final volume of 50 μL. The following conditions were used for the Amplification of the Arabinose insert: 98° C. for 2 minutes; 98° C. for 20 seconds (×30 cycles); 55° C. for 15 seconds (×30 cycles); 68° C. for 90 seconds (×30 cycles); and 68° C. for 1 minute. The PCR product was purified using QIAQUICK® PCR spin columns.

Purified PCR products were then used in another PCR amplification with primers SEQ ID NOs: 9 and 10. Each PCR reaction comprised: 10 μL of 5× PRIMESTAR® GXL Polymerase Buffer; 4 μL DNTPs; 1 μL of 10 μM Forward Primer; 1 μL of 10 μM Reverse Primer; 50 ng of the made PCR Product; 1 μL of PRIMESTAR® GXL Polymerase; and MILLI-Q® water to a final volume of 50 μL. The following conditions were used for the Amplification of the Arabinose insert: 98° C. for 2 minutes; 98° C. for 20 seconds (×30 cycles); 60° C. for 15 seconds (×30 cycles); 68° C. for 90 seconds (×30 cycles); and 68° C. for 1 minute. The PCR product was purified using QIAQUICK® PCR spin columns.

Purified PCR products were then used in another PCR using SEQ ID NOs: 9 and 10 as primers, in a reaction mixture comprising the follow (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of the made PCR Product; 1 μL of PRIMESTAR® GXL Polymerase; and MILLI-Q® water to a final volume of 50 μL. The following conditions were used for the Amplification of the Arabinose insert: 98° C. for 2 minutes; 98° C. for 20 seconds (×30 cycles); 60° C. for 15 seconds (×30 cycles); 68° C. for 90 seconds (×30 cycles); and 68° C. for 1 minute. The PCR product was purified using QIAQUICK® PCR spin columns.

100 ng of the digested and purified vector was prepared for a GIBSON ASSEMBLY® reaction. 58 ng of the amplified and purified promoter was also prepared for the reaction. No more than 0.1 pmols of total DNA were used in the GIBSON ASSEMBLY®. Vector DNA and Insert DNA were mixed with MILLI-Q® water to a final volume of 10 μL. 10 μL of 2×NEB HIFI Assembly Master mix (obtained from NEW ENGLAND BIOLABS®) was then added to the reaction mixture. The prepared GIBSON ASSEMBLY® reactions were then incubated in a thermal cycler at 50° C. for 1 hour. Following the incubation period, 8 μL of the reaction mixture were then transformed using NEB5α ultracompetent cells.

Transformed cells with the arabinose promoter cloned into the pET-Duet dual expression plasmid were used to amplify the plasmid DNA which was then harvested using the QIAGEN® Maxi Prep Purification system. The resulting plasmid was labeled as pBM123 to be used in cloning.

Cloning of pBM127, Vaccinia Virus Capping Enzyme in Dual Arabinose Promoting Expression System. SEQ ID NOs: 11 and 12 primers were used to amplify the D12 subunit for purposes of cloning into the second multiple cloning site of the arabinose promoting system in the following reaction mixture (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of D12 subunit Template; 1 μL of PRIMESTAR® GXL Polymerase; and MILLI-Q® water to a final volume of 50 μL. The following conditions were used for the Amplification of the D12 GIBSON ASSEMBLY® insert: 98° C. for 2 minutes; 98° C. for 20 seconds (×30 cycles); 55° C. for 15 seconds (×30 cycles); 68° C. for 90 seconds (×30 cycles); 55° C. for 15 seconds (×30 cycles); 68° C. for 120 seconds (×30 cycles); ad 68° C. for 1 minute. The PCR product was purified using QIAQUICK® PCR spin columns.

10 μg of pBM125 plasmid was digested using Nco1-HF restriction enzyme using the following reaction mixture (per reaction): 9 μL of CUTSMART® buffer; 2 μL of Nco1-HF; 10 μg of pBM125; and MILLI-Q® water to a final volume of 90 μL. Incubation was completed in a thermal cycler for 1 hour at 37° C. Antarctic Phosphatase was then added (10 μL/reaction of Antarctic Phosphatase Buffer & 2 μL/reaction of Antarctic Phosphatase). Reaction mixture was then incubated at 37° C. for 1 hour. The digested vector was then gel extracted and purified using the QIAGEN® gel extraction purification kit.

100 ng of the digested and purified vector was prepared for a GIBSON ASSEMBLY® reaction. 125 ng of the amplified and purified subunit was also prepared for the reaction. No more than 0.1 pmols of total DNA were used in the Gibson assembly. Vector DNA and Insert DNA were mixed with MILLI-Q® water to a final volume of 10 μL. 10 μL of 2×NEB HIFI Assembly Master mix (obtained from NEW ENGLAND BIOLABS®) was then added to the reaction mixture. The prepared GIBSON ASSEMBLY® reactions were then incubated in a thermal cycler at 50° C. for 15 minutes. Following the incubation period, 8 μL of the reaction mixture were then transformed using NEB5α ultracompetent cells. Transformed cells with the D12 subunit cloned in were used to amplify the plasmid DNA which was then harvested using the QIAGEN® Mini Prep Purification system. The resulting DNA was labeled as pBM127 and used for transformations into CLEARCOLI™ competent cells for protein expression.

Cloning of pBM135 for creation of E. coli Poly(A) Polymerase expression system. The PCR reaction mixture was prepared as follows with SEQ ID NOs: 15 and 16 as primers (volumes per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of Poly(A) Polymerase DNA; 1 μL of PRIMESTAR® GXL Polymerase. The PCR to amplify the Poly(A) Polymerase insert was completed as follows: 98° C. for 2 minutes; 98° C. for 20 seconds (×30 cycles); 55° C. for 15 seconds (×30 cycles); 68° C. for 90 seconds (×30 cycles); and 68° C. for 1 minute. The completed PCR was then purified using QIAQUICK® PCR spin columns.

Purified PCR products were then used in another PCR with SEQ ID NOs: 17 and 18 as primers. PCR reactions were prepared as follows (per reaction): 10 μL of 5× PRIMESTAR® GXL Polymerase Buffer; 4 μL DNTPs; 1 μL of 10 μM Forward Primer; 1 μL of 10 μM Reverse Primer; 50 ng of the made PCR Product; 1 μL of PRIMESTAR® GXL Polymerase; and MILL-Q® water to a final volume of 50 μL. The following conditions were used for the Amplification of the Poly(A) Polymerase insert: 98° C. for 2 minutes; 98° C. for 20 seconds (×30 cycles); 55° C. for 15 seconds (×30 cycles); 68° C. for 60 seconds (×30 cycles); 68° C. for 1 minute. The PCR product was purified using QIAQUICK® PCR spin columns.

20 μg of pBM98 was used in a double restriction digest using the enzymes: Nde1 and Nco1-HF using the following reaction recipe (per reaction): 90 μL of CUTSMART® buffer; 1 μL of Nco1-HF; 1 μL of Nde1; 20 μg of pBM98; and water to a final volume of 90 μL. The restriction digest was incubated in a thermal cycler for 2 hours at 37° C. Antarctic Phosphatase was then added (10 μL/reaction of Antarctic Phosphatase Buffer & 2 μL/reaction of Antarctic Phosphatase). Reaction mixture was then incubated at 37° C. for 1 hour. The digested vector was then gel extracted and purified using the QIAGEN® gel extraction purification kit.

100 ng of the digested and purified vector was prepared for a GIBSON ASSEMBLY® reaction. 63 ng of the amplified and purified Poly(A) Polymerase insert was also prepared for the reaction. No more than 0.1 pmols of total DNA were used in the GIBSON ASSEMBLY®. Vector DNA and Insert DNA were mixed with MILLI-Q® water to a final volume of 10 μL. 10 μL of 2×NEB HIFI Assembly Master mix (obtained from NEW ENGLAND BIOLABS®) was then added to the reaction mixture. The prepared GIBSON ASSEMBLY® reactions were then incubated in a thermal cycler at 50° C. for 15 minutes. Following the incubation period, 8 μL of the reaction mixture were then transformed using NEB5α ultracompetent cells.

Transformed cells with the E. coli Poly(A) Polymerase cloned into the Arabinose Promoted expression plasmid were used to amplify the plasmid DNA which was then harvested using the QIAGEN® Mini Prep Purification system.

Production of RNA Transcript from linearized DNA. This procedure is used for preparations of reactions for in vitro RNA transcript generation encoding a desired gene of interest for further downstream modifications. The list of equipment used in the process includes: 96 well thermal cycler; 10× transcription buffer; micropipette; NTPs (100 mM); 8-strip PCR tubes; inorganic pyrophosphatase; RNAse inhibitor (murine); Not1-HF enzyme; in-house purified T7 RNA polymerase; and QIAGEN® PCR Purification Kit.

To prepare the In Vitro Transcription (IVT) template, one obtains an aliquot of pRNI-GFP to prepare for restriction digest. 10 μg of template DNA were digested for every three IVT reactions being prepared using the following recipe (volumes per reaction): 5 μL of CUTSMART® Buffer; 2 μL of Not1-HF Restriction Enzyme; 10 μg of pRNI-GFP; nuclease free water to a final volume of 100 μL. Add the above contents into a 1.5 mL microcentrifuge tube labeled: Name: “pRNI-GFP Digest.” Incubate in a 37° C. incubator for 2 hours. Following incubation, digested DNA was removed and 500 μL of Buffer PB was added. The mixture of Buffer PB and digested DNA was transferred to a purple QIAGEN® PCR Purification column. The column spun in a tabletop centrifuge at max speed for 1 minute and flowthrough was discarded. 750 μL of Buffer PE went directly to the column, followed by a spin in the centrifuge for 1 minute at max speed. Again, flowthrough was discarded. The column was then spun dry at max speed for 3 minutes. A 1.5 mL microcentrifuge tube was labeled: “Digested pRNI-GFP.” The column went into the newly labeled microcentrifuge tube. 40 μL of nuclease free water was placed directly to the membrane. Following a 2 minute incubation at 37° C., the column was spun at max speed in centrifuge for 2 minutes. The elution fraction was analyzed using a spectrophotomer per manufacturer's instructions.

To prepare IVT reactions, one labels a PCR tube with the following: Name: “IVT Reaction.” The following tubes were obtained and kept on ice at all times: vial of in-house purified T7 RNA polymerase; vial of inorganic pyrophosphatase; vial of RNAse inhibitor (murine); tube of “pRNI-GFP Digest.” The following reagents were thawed at room temperature: 10× Reaction Buffer; ATP; CTP; UTP; and GTP. Add the following reagents into the labeled PCR tube in the following order: RNAse free water (≤20 μL); 10× reaction buffer (2 μL); 100 mM ATP (2 μL); 100 mM CTP (2 μL); 100 mM GTP (2 μL); 100 mM UTP (2 μL); template DNA (1 μg); T7 RNA Polymerase Mix (2 μL); RNAse inhibitor (1 μL); inorganic pyrophosphatase (2 μL). The final volume of one reaction should be 20 μL. The prepared PCR tube was briefly centrifuged, and then incubated at 37° C. in a thermal cycler overnight.

PURIFICATION OF IVT RNA TRANSCRIPTS. Following overnight incubation, the tubes were removed from thermal cycler and 100 μL of Buffer PB added to the reaction using a filtered p200 micropipette. It is typical at this point in the process that the reaction vessel will be cloudy following the overnight incubation. When adding Buffer PB there will be presence of translucent solid which should be mixed gently using the pipette.

The mixture was added directly to the membrane of a purple QIAGEN® PCR purification column. The column spun at max speed for 1 minute, and the flowthrough was discarded. 750 μL of Buffer PE was added to each column and spun at max for 1 minute. Flowthrough was discarded. The column spun dry at max speed for 3 minutes. A new 1.5 mL Microcentrifuge tube was labeled: “In Vitro Transcript.” The column was moved into the labeled microcentrifuge tube and 35 μL of RNAse free water was added directly to the membrane. The column spun at max speed for 2 minutes. The eluted RNA transcript was then analyzed in a spectrophotometer per manufacturer's instructions. Following spectrophotometer analysis, the purified RNA transcript should be kept on ice.

If the Capping reaction cannot be completed directly after the purification, any RNA transcripts should be stored at −80° C. Frozen RNA should not be stored for more than 1 week for best results.

Production of Capped RNA Transcript. The following components were stored at −20° C. prior to the start of work and kept on ice during reaction assembly: 10× Capping Buffer, 32 mM S-adenosyl Methionine (SAM), 10 mM guanosine triphosphate (GTP), RNAse inhibitor (murine), in-house purified vaccinia virus capping enzyme (VVCE). A microcentrifuge tube was labeled: “2 mM SAM.” SAM was diluted 1:16 by adding 30 μL of RNAse free water and 2 μL of the 32 mM stock into the labeled microcentrifuge tube. It is best to dilute SAM just prior to the reactions for highest capping efficiencies. Two new PCR strip tubes were labeled: “VVCE Capping Reaction.” 10 μg of RNA were added to each labeled tube using the purified RNA collected. RNAse free water was added to a final volume of 14 μL for each tube. The RNA and water mixture was denatured at 65° C. for 5 minutes in the thermal cycler. Immediately post-denaturation, the RNA was placed on ice for 5 minutes. The reagents were added to each reaction in the following order (volumes per reaction): 2 μL of 10× Capping Buffer; 1 μL of 10 mM GTP; 1 μL of 2 mM SAM; 1 μL of VVCE; and 1 μL of RNAse inhibitor. The contents were mixed gently using a pipette and spun down. Reactions were incubated in a thermal cycler for 1 hour at 37° C.

Following the 37° C. incubation, the tubes were removed from the thermal cycler and 100 μL of Buffer PB was added to each reaction using a filtered p200 micropipette. The contents of each reaction tube were carefully moved onto the membrane of a purple QIAGEN® PCR purification column. The column was centrifuged at max speed for 1 minute, and the flowthrough discarded. 750 μL of Buffer PE was added to each column and spun at max for 1 minute. After discarding the flowthrough, the column was dried by spinning at max speed for 3 minutes. Two new 1.5 mL microcentrifuge tubes were labeled: “Capped RNA Transcript.” The columns were placed in the labeled microcentrifuge tubes and 35 μL of RNAse free water was added directly to the membrane and incubated for 2 minutes. The product was collected by spinning the column at max speed for 2 minutes. The prepared RNA transcript was analyzed using the spectrophotomer per manufacturer's instructions. Following the SPEC, the purified RNA transcript was kept on ice and moved directly to the tailing reaction. If the tailing reaction cannot be completed directly after the purification, any RNA transcripts should be stored at −80° C. for the time being (no longer than 1 week for best results).

Production of poly-adenylated mRNA. The following components were stored at −20° C. prior to the start of work and kept on ice during reaction assembly: 10× Tailing Buffer; 10 mM adenosine triphosphate (ATP); RNAse inhibitor (murine); and in-house purified poly(a) polymerase. Two new PCR strip tubes were labeled: “Poly(A) Polymerase Tailing Reaction.” 10 μg of RNA were added to each labeled tube using the purified RNA collected. RNAse free water was added to a final volume of 14 μL for each tube. The reagents were added to each reaction in the following order: 2 μL of 10× tailing Buffer; 2 μL of 10 mM ATP; 1 μL of poly(A) polymerase; and 1 μL of RNAse inhibitor. The contents were mixed gently using a pipette and spun down. Reactions were incubated in a thermal cycler for 1 hour at 37° C.

Following the incubation period, the tubes were removed from the thermal cycler and 100 μL of Buffer PB added to each reaction using a filtered p200 micropipette. The mixtures were directly added to the membrane of a purple QIAGEN® PCR purification column. The column was centrifuged at max speed for 1 minute, and the flowthrough discarded. 750 μL of Buffer PE was added to the column, followed by a spin at max for 1 minute. After discarding the flowthrough, the column was spun dry at max speed for 3 minutes. The column was placed in a new 1.5 mL microcentrifuge tube and 35 μL of RNAse free water added directly to the membrane. The column was allowed to sit for 2 minutes, and then spun at max speed for 2 minutes. The prepared RNA transcript was then analyzed using the spectrophotomer per manufacturer's instructions. Following the SPEC, the purified RNA transcript should be kept on ice and proceed directly to the Transfection reactions. If the transfections cannot be completed directly after the purification, any RNA transcripts should be stored at −80° C.

The following discussion provides many example embodiments. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the concepts herein. The present disclosure, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1-16. (canceled)

17. A composition for in vitro synthesis of messenger RNA (mRNA), the composition comprising:
a. a first plasmid vector comprising an arabinose promoter linked to a nucleic acid sequence encoding T7 RNA polymerase, wherein the first plasmid vector comprises a nucleotide sequence that is at least 95% identical to SEQ ID NO: 19;
b. a second plasmid vector comprising at least one arabinose promoter linked to a nucleic acid sequence encoding vaccinia virus capping enzyme (VVCE), wherein the second plasmid vector comprises a nucleotide sequence that is at least 95% identical to SEQ ID NO: 20; and
c. a third plasmid vector comprising a UUG start codon, an arabinose promoter linked to a nucleic acid sequence encoding polyadenosine (poly(A)) polymerase, wherein the third plasmid vector comprises a nucleotide sequence that is at least 95% identical to SEQ ID NO: 21.

18. The composition of claim 17, wherein the first, second, and third plasmid vectors are viral vectors.

19. The composition of claim 17 wherein:
(a) a first plurality of competent bacterial cells comprises the first plasmid vector, and wherein the T7 RNA polymerase is expressed by the first plurality of competent bacterial cells and purified;
(b) a second plurality of competent bacterial cells comprises the second plasmid vector, and wherein the VVCE is expressed by the second plurality of competent bacterial cells and purified; and
(c) a third plurality of competent bacterial cells comprises the third plasmid vector, and wherein the poly(A) polymerase is expressed by the third plurality of competent bacterial cells and purified.

20. The composition of claim 19, wherein (a) a linearized plasmid DNA is added to the purified T7 RNA polymerase to produce RNA transcripts; (b) the RNA transcripts are added to the purified VVCE to produce capped RNA transcripts; and (c) the capped RNA transcripts are added to the purified poly(A) polymerase to produce polyadenylated capped RNA transcripts.

21. The composition of claim 19, wherein the first, second, and third plurality of bacterial competent cells are comprised of E. coli BL21 (DE3) competent cells.

22. The composition of claim 17, wherein the first plasmid vector comprises the nucleotide sequence of SEQ ID NO: 19.

23. The composition of claim 17, wherein the second plasmid vector comprises the nucleotide sequence of SEQ ID NO: 20.

24. The composition of claim 17, wherein the third plasmid vector comprises the nucleotide sequence of SEQ ID NO: 21.

25. A kit for use in in vitro synthesis of mRNA, the kit comprising:
a) a first plasmid vector comprising an arabinose promoter linked to a nucleic acid sequence encoding T7 RNA polymerase, wherein the first plasmid vector comprises a nucleotide sequence that is at least 95% identical to SEQ ID NO: 19;
b) a second plasmid vector comprising at least one arabinose promoter linked to a nucleic acid sequence encoding vaccinia virus capping enzyme (VVCE), wherein the second plasmid vector comprises a nucleotide sequence that is at least 95% identical to SEQ ID NO: 20;
c) a third plasmid vector comprising a UUG start codon, an arabinose promoter linked to a nucleic acid sequence encoding polyadenosine (poly(A)) polymerase, wherein the third plasmid vector comprises a nucleotide sequence that is at least 95% identical to SEQ ID NO: 21;
d) a first, second and third plurality of competent bacterial cells; and
e) directions for use thereof.

26. The kit of claim 25, wherein the first, second, and third plasmid vectors are viral vectors.

27. The kit of claim 25, wherein:
(a) the first plurality of competent bacterial cells comprises the first plasmid vector, and wherein the T7 RNA polymerase is expressed by the first plurality of competent bacterial cells and purified;
(b) the second plurality of competent bacterial cells comprises the second plasmid vector, and wherein the VVCE is expressed by the second plurality of competent bacterial cells and purified; and
(c) the third plurality of competent bacterial cells comprises the third plasmid vector, and wherein the poly(A) polymerase is expressed by the third plurality of competent bacterial cells and purified.

28. The kit of claim 27, wherein (a) a linearized plasmid DNA is added to the purified T7 RNA polymerase to produce RNA transcripts; (b) the RNA transcripts are added to the purified VVCE to produce capped RNA transcripts; and (c) the capped RNA transcripts are added to the purified poly(A) polymerase to produce polyadenylated capped RNA transcripts.

29. The kit of claim 25, wherein the first, second, and third plurality of competent bacterial cells are comprised of E. coli BL21 (DE3) competent cells.

30. The kit of claim 25, wherein the first plasmid vector comprises the nucleotide sequence of SEQ ID NO: 19.

31. The kit of claim 25, wherein the second plasmid vector comprises the nucleotide sequence of SEQ ID NO: 20.

32. The kit of claim 25, wherein the third plasmid vector comprises the nucleotide sequence of SEQ ID NO: 21.


SEQUENCE LISTING (26 sequences)

SEQ ID NO: 1 - DNA, 26 bp, synthetic construct
  source 1..26: mol_type = other DNA
ataatgtgcctgtcaaatggacgaag

SEQ ID NO: 2 - DNA, 25 bp, synthetic construct
  source 1..25: mol_type = other DNA
ctagcccaaaaaaacgggtatggag

SEQ ID NO: 3 - DNA, 60 bp, synthetic construct
  source 1..60: mol_type = other DNA
ccacgatgcgtccggcgtagaggatcgagatctataatgtgcctgtcaaatggacgaagc

SEQ ID NO: 4 - DNA, 56 bp, synthetic construct
  source 1..56: mol_type = other DNA
cttcttaaagttaaacaaaattatttctagactagcccaaaaaaacgggtatggag

SEQ ID NO: 5 - DNA, 60 bp, synthetic construct
  source 1..60: mol_type = other DNA
gtttaactttaagaaggagatatattcgaaatgcatcaccatcaccatcacgctagcatg

SEQ ID NO: 6 - DNA, 59 bp, synthetic construct
  source 1..59: mol_type = other DNA
cgaattcggatccgaattaattccgatatccatggttacgcgaacgcgaagtccgactc

SEQ ID NO: 7 - DNA, 59 bp, synthetic construct
  source 1..59: mol_type = other DNA
ggatcgagatcgatctcgatcccgcgaaatataatgtgcctgtcaaatggacgaagcag

SEQ ID NO: 8 - DNA, 65 bp, synthetic construct
  source 1..65: mol_type = other DNA
ctccttcttaaagttaaacaaaattatttctagactagcccaaaaaaacgggtatggagaaacag

SEQ ID NO: 9 - DNA, 63 bp, synthetic construct
  source 1..63: mol_type = other DNA
gcttaagtcgaacagaaagtaatcgtattgtacaataatgtgcctgtcaaatggacgaagcag

SEQ ID NO: 10 - DNA, 76 bp, synthetic construct
  source 1..76: mol_type = other DNA
catatgtatatctccttcttatacttaactaatatactaagatggctagcccaaaaaaacgggtatggagaaacag

SEQ ID NO: 11 - DNA, 57 bp, synthetic construct
  source 1..57: mol_type = other DNA
gtatattagttaagtataagaaggagatatacaatggacgagattgttaaaaacatt

SEQ ID NO: 12 - DNA, 57 bp, synthetic construct
  source 1..57: mol_type = other DNA
gtggccggccgatatccaattgagatctgccattacagtaataatttcaccaggcga

SEQ ID NO: 13 - DNA, 57 bp, synthetic construct
  source 1..57: mol_type = other DNA
gaaataattttgtttaactttaagaaggagatatacatgaaacaccatcaccatcac

SEQ ID NO: 14 - DNA, 57 bp, synthetic construct
  source 1..57: mol_type = other DNA
cgacctgcaggcgcgccgagctcgaattcgttaacgtttcgagaacacgtagacaac

SEQ ID NO: 15 - DNA, 29 bp, synthetic construct
  source 1..29: mol_type = other DNA
tgacactaccgaggtgtactatttttacc

SEQ ID NO: 16 - DNA, 18 bp, synthetic construct
  source 1..18: mol_type = other DNA
tgcggtaccctcacgacg

SEQ ID NO: 17 - DNA, 67 bp, synthetic construct
  source 1..67: mol_type = other DNA
gaaataattttgtttaactttaagaaggagatatacattgacactaccgaggtgtactatttttacc

SEQ ID NO: 18 - DNA, 71 bp, synthetic construct
  source 1..71: mol_type = other DNA
ctcgaattcggatccgaattaattccgatatctcagtggtgatggtgatggtgtgcggtaccctcacgacg

SEQ ID NO: 19 - DNA, 9347 bp, synthetic construct
  source 1..9347: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagtctagaaataattttgtttaactttaagaaggagatatattcgaaatgcatcaccatcaccatcacgctagcatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaccatggatatcggaattaattcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 20 - DNA, 11338 bp, synthetic construct
  source 1..11338: mol_type = other DNA
attcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaatataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagtctagaaataattttgtttaactttaagaaggagatatacatgaaacaccatcaccatcaccaccccatgtcagactacgatatcccaaccactgaaaacttatacttccaaggggcaatggacgcaaacgtcgtaagcagttctaccattgcgacctacattgacgcactggcaaaaaatgcatcggaattagagcagcgctctaccgcctatgaaattaacaatgagttagaattagtttttattaagcctccgttaattaccttaaccaatgtagtcaatattagtactattcaagagtctttcatccgtttcactgtaactaacaaggaaggggtgaagatccgcacgaagattccattgtcgaaagtacacggacttgatgtaaagaacgttcaattggtagatgcaattgacaatattgtatgggaaaaaaagtctcttgtgaccgaaaaccgtttacataaagagtgtctgctgcgtttatctacggaggagcgccacatctttttggattacaagaaatacggatcttctatccgtctggaattggtcaacttaattcaggccaagactaaaaatttcacaattgatttcaaattaaaatactttttgggctcaggtgcgcagtctaagtcgagcttattgcatgctatcaaccatccgaaaagtcgccccaacacctctctggagattgagtttacgccacgcgataacgagacagtgccgtatgacgaattaattaaagaattgaccacgttaagccgccatattttcatggcgtcgccagagaatgttatccttagtcccccaattaatgcgccaattaagaccttcatgttacccaagcaagacatcgtgggtctggacttagagaacttgtacgcagtcaccaaaacggatggtattccaatcactattcgcgtaaccagtaacggactttattgctacttcacgcatctgggttacatcattcgttacccggttaaacgtatcatcgattctgaggtcgtggtattcggcgaggcagtgaaagataaaaactggactgtataccttattaaacttatcgagccggtaaatgcgattaatgatcgtttagaagaaagcaagtatgtggaatctaagcttgtcgatatctgtgatcgtatcgtgttcaagtcgaaaaagtatgaggggcccttcaccactactagtgaggtagtagatatgcttagtacgtatttgccaaaacaaccagaaggtgtcattctgttctactccaaaggccctaagtccaatattgacttcaaaattaaaaaggagaatacaattgaccaaacggcaaatgttgtatttcgctacatgtcgtctgaacccattatcttcggggaatcaagcatctttgtagagtacaagaagttctcgaatgacaagggttttccgaaagagtacggtagtggaaagatcgtgctttacaacggggtgaactaccttaataatatctattgtttggagtatattaacacgcacaatgaggtgggaatcaaatcggtggtcgtacctattaagttcatcgcggaatttttggtgaatggggagattttgaaaccacgtatcgataaaacgatgaagtacattaactctgaggactactacggtaaccagcataacattatcgtagagcatttgcgcgaccaatccatcaaaatcggggacatttttaatgaagacaaactttctgacgtaggccaccagtatgcaaataatgataagtttcgtcttaatcccgaggtgtcttattttactaataaacgcacgcgcggcccgttaggaattttaagcaactacgttaagaccttgctgatctctatgtattgcagtaaaacgttcttagacgattccaataagcgcaaagtacttgctattgacttcggtaatggggctgacctggagaagtatttttacggtgagatcgctttgcttgtggctaccgacccggacgccgacgccatcgcccgtggcaatgagcgctataacaaattgaattctggtattaagactaaatactataagtttgattatattcaagagacgatccgctcggatacgtttgtttcgtccgtacgcgaagtattctatttcgggaaatttaatatcattgactggcaatttgcaattcattacagcttccacccccgtcactatgcgacggtcatgaacaatttgtccgaattaacggcttcaggcggcaaggtgttgattacaacaatggatggagataaattatccaaattaaccgataaaaagacattcatcatccacaagaacttaccgagctcggaaaattatatgtcagtagaaaaaattgcggatgatcgtatcgtggtttacaatccgagcaccatgtccacgcctatgacggaatacatcattaagaaaaacgacattgtacgcgtattcaacgaatatggtttcgttcttgtcgacaacgttgactttgctaccatcatcgaacgttctaaaaagtttattaatggtgccagtaccatggaagaccgtccatccacccgcaatttctttgagcttaatcgcggtgcaatcaaatgcgaaggacttgatgtggaagatcttcttagttattacgttgtctacgtgttctcgaaacgttaacgaattcgagctcggcgcgcctgcaggtcgacaagcttgcggccgcataatgcttaagtcgaacagaaagtaatcgtattgtacaataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagccatcttagtatattagttaagtataagaaggagatatacaatggacgagattgttaaaaacattcgcgagggaacgcacgtcttgttacccttctacgagactttaccggagttgaacttgtctctgggaaagagtcccttaccgagtttagagtacggcgcaaactactttctgcaaattagtcgcgttaatgatctgaatcgtatgcccacagatatgttgaagctgtttacccacgacattatgctgccggaatctgacttagataaggtatacgaaatcctgaagatcaatagcgttaaatattatgggcgctcgactaaagcggatgcggttgtggcggatctgtccgcgcgcaataaattattcaagcgcgaacgcgatgcaatcaaaagtaataaccatcttaccgaaaataatctgtatatttccgactataaaatgttgacattcgacgtttttcgccccttgtttgactttgtgaacgagaaatactgcattatcaagctgccgactttattcggacgtggcgtaattgacaccatgcgtatctattgttcgttgtttaagaacgtccgtttgcttaagtgtgtgtccgatagctggttaaaggacagcgcgatcatggtggcttctgatgtgtgtaagaaaaatcttgatctgttcatgagccacgtcaaatctgtcactaaaagcagcagttggaaggacgttaacagcgttcagttctcgatcttgaacaacccagtggacactgaattcattaacaaatttcttgaattttcaaaccgcgtatacgaggcattgtattatgtacactctcttctttattcgtcaatgacgagtgattcaaagagtatcgaaaacaaacaccaacgtcgcctggtgaaattattactgtaatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 21 - DNA, 8096 bp, synthetic construct
  source 1..8096: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagtctagaaataattttgtttaactttaagaaggagatatacattgacactaccgaggtgtactatttttacccgagtcgctaatttttgccgcaaggtgctaagccgcgaggaaagcgaggctgaacaggcagtcgcccgtccacaggtgacggtgatcccgcgtgagcagcatgctatttcccgcaaagatatcagtgaaaatgccctgaaggtaatgtacaggctcaataaagcgggatacgaagcctggctggttggcggcggcgtgcgcgacctgttacttggcaaaaagccgaaagattttgacgtaaccactaacgccacgcctgagcaggtgcgcaaactgttccgtaactgccgcctggtgggtcgccgtttccgtctggctcatgtaatgtttggcccggagattatcgaagttgcgaccttccgtggacaccacgaaggtaacgtcagcgaccgcacgacctcccaacgcgggcaaaacggcatgttgctgcgcgacaacattttcggctccatcgaagaagacgcccagcgccgcgatttcactatcaacagcctgtattacagcgtagcggattttaccgtccgtgattacgttggcggcatgaaggatctgaaggacggcgttatccgtctgattggtaacccggaaacgcgctaccgtgaagatccggtacgtatgctgcgcgcggtacgttttgccgccaaattgggtatgcgcatcagcccggaaaccgcagaaccgatccctcgcctcgctaccctgctgaacgatatcccaccggcacgcctgtttgaagaatcgcttaaactgctacaagcgggctacggttacgaaacctataagctgttgtgtgaatatcatctgttccagccgctgttcccgaccattacccgctacttcacggaaaatggcgacagcccgatggagcggatcattgaacaggtgctgaagaataccgatacgcgtatccataacgatatgcgcgtgaacccggcgttcctgtttgccgccatgttctggtacccactgctggagacggcacagaagatcgcccaggaaagcggcctgacctatcacgacgctttcgcgctggcgatgaacgacgtgctggacgaagcctgccgttcactggcaatcccgaaacgtctgacgacattaacccgcgatatctggcagttgcagttgcgtatgtcccgtcgtcagggtaaacgcgcatggaaactgctggagcatcctaagttccgtgcggcttatgacctgttggccttgcgagctgaagttgagcgtaacgctgaactgcagcgtctggtgaaatggtggggtgagttccaggtttccgcgccaccagaccaaaaagggatgctcaacgagctggatgaagaaccgtcaccgcgtcgtcgtactcgtcgtccacgcaaacgcgcaccacgtcgtgagggtaccgcacaccatcaccatcaccactgagatatcggaattaattcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 22 - DNA, 2679 bp, synthetic construct
  source 1..2679: mol_type = other DNA
atgcatcaccatcaccatcacgctagcatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 23 - DNA, 2613 bp, synthetic construct
  source 1..2613: mol_type = other DNA
atgaaacaccatcaccatcaccaccccatgtcagactacgatatcccaaccactgaaaacttatacttccaaggggcaatggacgcaaacgtcgtaagcagttctaccattgcgacctacattgacgcactggcaaaaaatgcatcggaattagagcagcgctctaccgcctatgaaattaacaatgagttagaattagtttttattaagcctccgttaattaccttaaccaatgtagtcaatattagtactattcaagagtctttcatccgtttcactgtaactaacaaggaaggggtgaagatccgcacgaagattccattgtcgaaagtacacggacttgatgtaaagaacgttcaattggtagatgcaattgacaatattgtatgggaaaaaaagtctcttgtgaccgaaaaccgtttacataaagagtgtctgctgcgtttatctacggaggagcgccacatctttttggattacaagaaatacggatcttctatccgtctggaattggtcaacttaattcaggccaagactaaaaatttcacaattgatttcaaattaaaatactttttgggctcaggtgcgcagtctaagtcgagcttattgcatgctatcaaccatccgaaaagtcgccccaacacctctctggagattgagtttacgccacgcgataacgagacagtgccgtatgacgaattaattaaagaattgaccacgttaagccgccatattttcatggcgtcgccagagaatgttatccttagtcccccaattaatgcgccaattaagaccttcatgttacccaagcaagacatcgtgggtctggacttagagaacttgtacgcagtcaccaaaacggatggtattccaatcactattcgcgtaaccagtaacggactttattgctacttcacgcatctgggttacatcattcgttacccggttaaacgtatcatcgattctgaggtcgtggtattcggcgaggcagtgaaagataaaaactggactgtataccttattaaacttatcgagccggtaaatgcgattaatgatcgtttagaagaaagcaagtatgtggaatctaagcttgtcgatatctgtgatcgtatcgtgttcaagtcgaaaaagtatgaggggcccttcaccactactagtgaggtagtagatatgcttagtacgtatttgccaaaacaaccagaaggtgtcattctgttctactccaaaggccctaagtccaatattgacttcaaaattaaaaaggagaatacaattgaccaaacggcaaatgttgtatttcgctacatgtcgtctgaacccattatcttcggggaatcaagcatctttgtagagtacaagaagttctcgaatgacaagggttttccgaaagagtacggtagtggaaagatcgtgctttacaacggggtgaactaccttaataatatctattgtttggagtatattaacacgcacaatgaggtgggaatcaaatcggtggtcgtacctattaagttcatcgcggaatttttggtgaatggggagattttgaaaccacgtatcgataaaacgatgaagtacattaactctgaggactactacggtaaccagcataacattatcgtagagcatttgcgcgaccaatccatcaaaatcggggacatttttaatgaagacaaactttctgacgtaggccaccagtatgcaaataatgataagtttcgtcttaatcccgaggtgtcttattttactaataaacgcacgcgcggcccgttaggaattttaagcaactacgttaagaccttgctgatctctatgtattgcagtaaaacgttcttagacgattccaataagcgcaaagtacttgctattgacttcggtaatggggctgacctggagaagtatttttacggtgagatcgctttgcttgtggctaccgacccggacgccgacgccatcgcccgtggcaatgagcgctataacaaattgaattctggtattaagactaaatactataagtttgattatattcaagagacgatccgctcggatacgtttgtttcgtccgtacgcgaagtattctatttcgggaaatttaatatcattgactggcaatttgcaattcattacagcttccacccccgtcactatgcgacggtcatgaacaatttgtccgaattaacggcttcaggcggcaaggtgttgattacaacaatggatggagataaattatccaaattaaccgataaaaagacattcatcatccacaagaacttaccgagctcggaaaattatatgtcagtagaaaaaattgcggatgatcgtatcgtggtttacaatccgagcaccatgtccacgcctatgacggaatacatcattaagaaaaacgacattgtacgcgtattcaacgaatatggtttcgttcttgtcgacaacgttgactttgctaccatcatcgaacgttctaaaaagtttattaatggtgccagtaccatggaagaccgtccatccacccgcaatttctttgagcttaatcgcggtgcaatcaaatgcgaaggacttgatgtggaagatcttcttagttattacgttgtctacgtgttctcgaaacgttaa

SEQ ID NO: 24 - DNA, 864 bp, synthetic construct
  source 1..864: mol_type = other DNA
atggacgagattgttaaaaacattcgcgagggaacgcacgtcttgttacccttctacgagactttaccggagttgaacttgtctctgggaaagagtcccttaccgagtttagagtacggcgcaaactactttctgcaaattagtcgcgttaatgatctgaatcgtatgcccacagatatgttgaagctgtttacccacgacattatgctgccggaatctgacttagataaggtatacgaaatcctgaagatcaatagcgttaaatattatgggcgctcgactaaagcggatgcggttgtggcggatctgtccgcgcgcaataaattattcaagcgcgaacgcgatgcaatcaaaagtaataaccatcttaccgaaaataatctgtatatttccgactataaaatgttgacattcgacgtttttcgccccttgtttgactttgtgaacgagaaatactgcattatcaagctgccgactttattcggacgtggcgtaattgacaccatgcgtatctattgttcgttgtttaagaacgtccgtttgcttaagtgtgtgtccgatagctggttaaaggacagcgcgatcatggtggcttctgatgtgtgtaagaaaaatcttgatctgttcatgagccacgtcaaatctgtcactaaaagcagcagttggaaggacgttaacagcgttcagttctcgatcttgaacaacccagtggacactgaattcattaacaaatttcttgaattttcaaaccgcgtatacgaggcattgtattatgtacactctcttctttattcgtcaatgacgagtgattcaaagagtatcgaaaacaaacaccaacgtcgcctggtgaaattattactgtaa

SEQ ID NO: 25 - DNA, 1437 bp, synthetic construct
  source 1..1437: mol_type = other DNA
ttgacactaccgaggtgtactatttttacccgagtcgctaatttttgccgcaaggtgctaagccgcgaggaaagcgaggctgaacaggcagtcgcccgtccacaggtgacggtgatcccgcgtgagcagcatgctatttcccgcaaagatatcagtgaaaatgccctgaaggtaatgtacaggctcaataaagcgggatacgaagcctggctggttggcggcggcgtgcgcgacctgttacttggcaaaaagccgaaagattttgacgtaaccactaacgccacgcctgagcaggtgcgcaaactgttccgtaactgccgcctggtgggtcgccgtttccgtctggctcatgtaatgtttggcccggagattatcgaagttgcgaccttccgtggacaccacgaaggtaacgtcagcgaccgcacgacctcccaacgcgggcaaaacggcatgttgctgcgcgacaacattttcggctccatcgaagaagacgcccagcgccgcgatttcactatcaacagcctgtattacagcgtagcggattttaccgtccgtgattacgttggcggcatgaaggatctgaaggacggcgttatccgtctgattggtaacccggaaacgcgctaccgtgaagatccggtacgtatgctgcgcgcggtacgttttgccgccaaattgggtatgcgcatcagcccggaaaccgcagaaccgatccctcgcctcgctaccctgctgaacgatatcccaccggcacgcctgtttgaagaatcgcttaaactgctacaagcgggctacggttacgaaacctataagctgttgtgtgaatatcatctgttccagccgctgttcccgaccattacccgctacttcacggaaaatggcgacagcccgatggagcggatcattgaacaggtgctgaagaataccgatacgcgtatccataacgatatgcgcgtgaacccggcgttcctgtttgccgccatgttctggtacccactgctggagacggcacagaagatcgcccaggaaagcggcctgacctatcacgacgctttcgcgctggcgatgaacgacgtgctggacgaagcctgccgttcactggcaatcccgaaacgtctgacgacattaacccgcgatatctggcagttgcagttgcgtatgtcccgtcgtcagggtaaacgcgcatggaaactgctggagcatcctaagttccgtgcggcttatgacctgttggccttgcgagctgaagttgagcgtaacgctgaactgcagcgtctggtgaaatggtggggtgagttccaggtttccgcgccaccagaccaaaaagggatgctcaacgagctggatgaagaaccgtcaccgcgtcgtcgtactcgtcgtccacgcaaacgcgcaccacgtcgtgagggtaccgcacaccatcaccatcaccactga

SEQ ID NO: 26 - DNA, 1389 bp, synthetic construct
  source 1..1389: mol_type = other DNA
atgcaccatcaccatcaccacaaggtgctaagccgcgaggaaagcgaggctgaacaggcagtcgcccgtccacaggtgacggtgatcccgcgtgagcagcatgctatttcccgcaaagatatcagtgaaaatgccctgaaggtaatgtacaggctcaataaagcgggatacgaagcctggctggttggcggcggcgtgcgcgacctgttacttggcaaaaagccgaaagattttgacgtaaccactaacgccacgcctgagcaggtgcgcaaactgttccgtaactgccgcctggtgggtcgccgtttccgtctggctcatgtaatgtttggcccggagattatcgaagttgcgaccttccgtggacaccacgaaggtaacgtcagcgaccgcacgacctcccaacgcgggcaaaacggcatgttgctgcgcgacaacattttcggctccatcgaagaagacgcccagcgccgcgatttcactatcaacagcctgtattacagcgtagcggattttaccgtccgtgattacgttggcggcatgaaggatctgaaggacggcgttatccgtctgattggtaacccggaaacgcgctaccgtgaagatccggtacgtatgctgcgcgcggtacgttttgccgccaaattgggtatgcgcatcagcccggaaaccgcagaaccgatccctcgcctcgctaccctgctgaacgatatcccaccggcacgcctgtttgaagaatcgcttaaactgctacaagcgggctacggttacgaaacctataagctgttgtgtgaatatcatctgttccagccgctgttcccgaccattacccgctacttcacggaaaatggcgacagcccgatggagcggatcattgaacaggtgctgaagaataccgatacgcgtatccataacgatatgcgcgtgaacccggcgttcctgtttgccgccatgttctggtacccactgctggagacggcacagaagatcgcccaggaaagcggcctgacctatcacgacgctttcgcgctggcgatgaacgacgtgctggacgaagcctgccgttcactggcaatcccgaaacgtctgacgacattaacccgcgatatctggcagttgcagttgcgtatgtcccgtcgtcagggtaaacgcgcatggaaactgctggagcatcctaagttccgtgcggcttatgacctgttggccttgcgagctgaagttgagcgtaacgctgaactgcagcgtctggtgaaatggtggggtgagttccaggtttccgcgccaccagaccaaaaagggatgctcaacgagctggatgaagaaccgtcaccgcgtcgtcgtactcgtcgtccacgcaaacgcgcaccacgtcgtgagggtaccgcatga